(19) United States
(12) Patent Application Publication
(10) Pub. No.: US 2024/0408055 A1
(43) Pub. Date: Dec. 12, 2024

(54) USE OF ANISOMELIC ACID IN PREPARATION OF PHARMACEUTICAL COMPOSITION FOR INHIBITING INFECTION AND REPLICATION OF SARS-COV-2 AND VARIANTS

(71) Applicants: Peking University Shenzhen Graduate School, Shenzhen City (CN); CNS BIOTEK CORP., Taichung City (TW); Gansu Evergreen Pharmaceuticals Co., Ltd., Lanzhou City (CN)

(72) Inventors: Zhen Yang, Shenzhen City (CN); Yew-Min Tzeng, Taichung City (TW); Jun-Min Quan, Shenzhen City (CN); Qing Chang, Lanzhou City (CN); Chi-Tai Yeh, Taipei City (TW)

(73) Assignees: Peking University Shenzhen Graduate School, Shenzhen City, GD (CN); CNS BIOTEK CORP., Taichung City, TW (TW); Gansu Evergreen Pharmaceuticals Co.,Ltd., Lanzhou City (CN)

(21) Appl. No.: 18/702,340

(22) PCT Filed: Oct. 21, 2021
(86) PCT No.: PCT/CN2021/125342

§ 371 Date: Apr. 18, 2024

(51) Int. Cl. A61K 31/365; A61K 36/53; A61P 31/14

(52) U.S. Cl. CPC A61K 31/365; A61K 36/53; A61P 31/14

(57) ABSTRACT

The present invention relates to a use of a natural compound Anisomelic acid extracted from Anisomeles indica O. Kuntze in the preparation of pharmaceutical compositions for inhibiting the infection and replication of novel coronaviruses and the mutant strains (SARS-CoV-2 variants) thereof, the Anisomelic acid is a compound comprising a chemical structural Formula I, the pharmaceutical composition includes a safe and effective amount of Anisomelic acid, that is, the pharmaceutical composition is a combination of a safe and effective amount of Anisomelic acid and its pharmaceutically acceptable salt or carrier thereof.

### CROSS-REFERENCE TO RELATED APPLICATION

This application is a national stage application, filed under 35 U.S.C. § 371, of International Patent Application No. PCT/CN2021/125342, filed on Oct. 21, 2021, which is incorporated by reference herein in its entirety.

### FIELD OF THE INVENTION

The present invention is related to an Anisomelic acid, a natural compound of Anisomeles indica O. Kuntze, for competitively inhibiting the binding of novel coronavirus to human Neuropilin-1 (NRP1) receptors, inhibiting the integration of cellular cathepsin (Cathepsin B/L) into the virus, inhibiting the transmembrane serine protease (TMPRSS2) to reduce virus invasion and infection, and inhibiting the main protease of novel coronavirus (Main protease, 3C-like protease; Mpro, 3CLpro) from replicating the virus, etc., for use in pharmaceutical composition. The present invention is particularly related to a pharmaceutical composition suitable for inhibiting the infection of host cells by coronaviruses and inhibiting the replication of coronaviruses in host cells, the pharmaceutical composition including Anisomelic acid, or isomers of Anisomelic acid, or derivatives of Anisomelic acid.

### BACKGROUND OF THE INVENTION

Since 2002 to the present, there have been several outbreaks of highly transmissible pathogenic β coronaviruses, such as SARS and MERS. Since the end of 2019, COVID-19, a disease caused by novel coronavirus SARS-CoV-2, has triggered a serious public health crisis around the world. At present, there are a series of highly infectious or highly pathogenic variant strains, which have a considerable and continuing concern on both vaccine prevention and drug treatment.

Anisomelic acid is a natural diterpene compound extracted from Anisomeles indica O. Kuntze. The content of Anisomelic acid in the whole plant of Anisomeles indica O. Kuntze is generally about 70 to 100 ppm of the dry weight of the plant. Anisomeles indica O. Kuntze is a commonly used herbal medicine among the people in Taiwan. It is also known as Hakka grass (Meizhou Jiaoling, Guangdong), golden sword grass, wrinkled giant hyssop Hemigraphis alternata, and Epimeredi indicus. Taiwan Ministry of Health and Welfare has included Anisomeles indica O. Kuntze in the list of materials available for food, and the whole plant is edible.

For more than 20 years, the inventor team has been engaged in breeding and cultivation of Anisomeles indica O. Kuntze (GenBank: GU726292) for a long time, and continues to conduct a series of research on the whole plant extract of Anisomeles indica O. Kuntze grown at ZiXiu Farm in Yu-li town, Hualien, Taiwan, especially focusing on purification and preparation of a series of natural substances contained in Anisomeles indica O. Kuntze, specifically carries out extraction, isolation, purification, analysis and identification, as well as anti-inflammation, anti-influenza virus, anti-Helicobacter pylori, anti-fatigue, anti-allergy, anti-asthma, anti-cancer, anti-cancer stem cells on the pharmacological effects research.

In 2014, the inventor team completed a comparative test experiment on the alcoholic extract and its series of purified products of Anisomeles indica O. Kuntze, comparing with the oral drug “Tamiflu” (Roche Pharmaceuticals) for the treatment of type A and type B influenza, and found that Anisomeles indica O. Kuntze extract and its series of purified products have good effects on inhibiting influenza viruses.

In a series of anti-cancer research, the inventor team explored the molecular docking simulation of the series natural substances of Anisomeles indica O. Kuntze and their interaction with cellular targets. In the results of series simulation, the inventor team discovered that Anisomelic acid or its oxidized derivative, Ovatodiolide, could inhibit angiogenesis by competing with Vascular Endothelial Growth Factor (VEGF) for binding to the receptor of human Neuropilin-1 (NRP1) and could lead to the suppression of tumor proliferation. The said Ovatodiolide is a compound with a formula II structure. Furthermore, the inventor team learned from two important academic research reports published in the current issue of Science journal on Nov. 13, 2020 which revealed that human Neuropilin-1 (NRP1) is a spike glycoprotein receptor on the surface of the novel coronavirus (SARS-CoV-2), this is the second receptor found in human cells in addition to angiotensin-converting enzyme-2 (ACE2) receptor that has been recognized in academia. this finding inspired the inventor for a series of studies and experimentally exploration of the development of Anisomelic acid and its oxidized derivative, Ovatodiolide, as a competitive drug to inhibit the infection of novel coronavirus in human through the human Neuropilin-1 (NRP1) receptor in human body.

The Genomics Research Center, Academia Sinica in Taiwan screened the anti-malaria drug Mefloquine, anti-HIV drug Nelfinavir, Chinese herbal medicine Ganoderma lucidum polysaccharide RF3, peppermint extract, and perilla extract and so on from 2,885 drugs approved by the U.S. Food and Drug Administration (FDA), 190 Chinese herbal medicines, and anti-SARS compounds synthesized in the past. After conducting oral administration experiments on hamster for three days (drug dosage: 30 mg/kg/day; extract dosage: 200 mg/kg/day), it was observed that the virus load could be reduced by up to 10 times compared with receiving water only. The key to the aforementioned antiviral drugs is to block the replication process of virus. However, due to Chinese herbal medicine is composed of multiple ingredients, its mechanism is unclear, and further research of the mechanism is still needed (PNAS Feb. 2, 2021, 118 (5) e2021579118). In view of the fact that Anisomeles indica O. Kuntze is also commonly known as Hemigraphis alternata, and the tests and findings reported above did not disclose the potential of Anisomeles indica O. Kuntze extract, Anisomelic acid or its oxidized derivatives Ovatodiolide against novel coronavirus, it prompted the motivation of the research team to initiate the investigation of Anisomelic acid and Ovatodiolide inhibiting the infection or replication of novel coronavirus.

In recent years, the inventor team has promoted the cultivation of Hakka grass at Chang's farm in Longnan, Gansu, resulting in abundant harvests. This effort ensures a steady supply of natural substances, including Anisomelic acid, which is very helpful for the planning and executing of the present invention. Notably important, the inventor team has recently completely developed an asymmetric synthesis process for the preparation of large quantities of optically pure Anisomelic acid, and the favorable conditions have been specifically created for in-depth research on the inhibition of novel coronavirus by the natural substances Anisomelic acid and the derivatives of Anisomelic acid contained in Anisomeles indica O. Kuntze.

### DETAILED DESCRIPTION OF THE INVENTION

The present invention is based on Anisomelic acid, a natural substance of Anisomeles indica O. Kuntze, which can be used to inhibit the invasion of host cells by novel coronavirus (SARS-CoV-2), to inhibit viral replication, and even to treat or prevent viral infectious diseases, especially pneumonia caused by novel coronavirus (or severe special infectious pneumonia, COVID-19). In particular, the present invention provides a method for inhibiting the invasion of the host by novel coronavirus, inhibiting viral replication, and even treating or preventing viral infectious diseases, comprising administering a pharmaceutical composition comprises a safe and effective amount of diterpenoid natural substance of Anisomeles indica O. Kuntze, Anisomelic acid, or a safe and effective amount of structural isomer of Anisomelic acid, or a safe and effective amount of oxidized derivative of Anisomelic acid, Ovatodiolide, or a safe and effective amount of a structural isomer of Ovatodiolide, with a combination of its pharmaceutically acceptable salts or carriers.

As show in FIG. 1, the Anisomelic acid of the present invention has a chemical structure as shown in Formula I.

As show in FIG. 2, the Ovatodiolide of the present invention has a chemical structural formula as shown in Formula II.

In a series of anti-novel coronavirus researches, the present invention explores the molecular docking simulation of Anisomelic acid or its oxidized derivative, Ovatodiolide, with human cell neuropil protein-1 (NRP1), it is found that Anisomelic acid or its oxidized derivative, Ovatodiolide, can competitively inhibit the binding of the spike glycoprotein on the surface of novel coronavirus to the NRP1 receptor by competing with the spike glycoprotein molecule on the surface of novel coronavirus for its binding to the NRP1 receptor in human cells, so as to achieve the effect of inhibiting novel coronavirus from infecting human cells via NRP1.

The present invention focuses on the co-receptors of Anisomelic acid or its oxidized derivative, Ovatodiolide, and novel coronavirus invading host cells, endosomal cathepsins (Cathepsin B, Cathepsin L) and transmembrane serine proteases (TMPRSS2), etc., to carry out individually relevant molecular docking simulations exploration and biochemical analyzes and inhibition experiments. The present invention has confirmed that Anisomelic acid or Ovatodiolide has the ability to inhibit cathepsin B/L and can inhibit the enzyme activity of TMPRSS2, etc., it achieves the efficiency of inhibiting the integration of viruses into host cells via cathepsin B/L or via transmembrane serine proteases. Anisomelic acid or Ovatodiolide is an effective inhibitor against the infection of novel coronavirus (SARS-CoV-2).

The present invention conducted the exploration of molecular docking simulations of Anisomelic acid or its oxidized derivative Ovatodiolide with the main protease (Mpro) of novel coronavirus, and a good combination of Anisomelic acid or Ovatodiolide with Mpro was found. It showed that Anisomelic acid or Ovatodiolide has the potential to inhibit the activity of Mpro (3C-like protease; 3CLpro), and it was confirmed by biochemical analysis experiments, an appropriate amount of Anisomelic acid or Ovatodiolide can indeed inhibit the Mpro activity of novel coronavirus and achieve the effect of inhibiting novel coronavirus replication.

Most of the mutations of novel coronavirus strains currently circulating in the world are located at the receptor-binding domain (RBD position) of the spike protein, thereby adapting to the ability to bind to the host's ACE2 receptor; The binding site between novel coronavirus spike protein S1 (SARS-CoV-2 spike protein S1) and the human NRP1 receptor, which is competitively inhibited by Anisomelic acid or Ovatodiolide, since it involves the Furin cleavage site and belongs to highly conserved site, according to statistics from the Global Initiative on Sharing All Influenza Data (GISIAD), the conservation of this target is 100%. From the above results and observations, it is known that both Anisomelic acid and Ovatodiolide have the ability to competitively inhibit the binding site of SARS-CoV-2 spike S1 and human NRP1 receptor and have a broad application value across virus strains in inhibiting viral infection and invasion of host.

The present invention specifically implements the novel coronavirus pseudovirus activity inhibition detecting system developed by the laboratory of Professor Zhang Linqi, the director of the AIDS Comprehensive Research Center of Tsinghua University, Beijing, to specifically evaluate whether Anisomelic acid or its oxidized derivative, Ovatodiolide, blocks infection of host cells by novel coronavirus. The experimental results showed that both Anisomelic acid and Ovatodiolide have the similar inhibitory effects on viral infections (such as SARS-CoV-2 variants) at the micromolar level with Remdesivir.

The present invention was specially commissioned by Nantong WuXi AppTec Co., Ltd., and was carried out professionally and concretely through the company's US biological laboratory; K18-hACE2 transgenic coronavirus-susceptible mice were used to carry out the anti-SARS-CoV-2 animal experiments planned by the inventor's team. The experimental results showed that: in addition to the obvious changes in the body weight of the mice in the placebo group after challenge, the body weight changes of the tested Anisomelic acid and its oxidized derivative, Ovatodiolide, were basically similar to the changes in the body weight of mice caused by Remdesivir, which can be said to be the safety of the three compounds were similar. At the same time, observing the changes in coronavirus infection titers in lung of mice administered with Anisomelic acid, Ovatodiolide, or Remdesivir. The results showed that the two tested compounds, Anisomelic acid and Ovatodiolide, showedsimilar changes in virus infection titers in mice caused by Remdesivir, and both showed preventive and therapeutic effects. Among them, Anisomelic acid showed better preventive and therapeutic effects. The above results show that Anisomelic acid and Ovatodiolide have the potential to be developed as oral anti-novel coronavirus drugs.

The term “novel coronavirus” in the present invention refers to SARS-CoV-2 that can cause severe special infectious pneumonia (COVID-19). It is an enveloped positive-stranded single-stranded RNA virus which belongs to the family of Coronaviridae, genus of beta coronavirus genus, severe acute respiratory syndrome-related coronavirus species, including related variant strains, etc.

The term “severe special infectious pneumonia (COVID-19)” in the present invention refers to the fatal pneumonia caused by the novel coronavirus (SARS-CoV-2) invading the human body. The novel coronavirus uses the spike glycoprotein on the surface of the coronavirus to recognize and bind to receptors such as angiotensin-converting enzyme-2 (ACE2) or neurocilidin-1 (NRP1) on the cell surface to infect normal cells in the human body; One possible pathogenic mechanism is that when the virus invades the body, the immune cells in the body act violently, triggering an immune storm in the body, releasing a large number of free radicals (such as peroxide free radicals) and denaturing proteins, DNA damage, and excessive production of cytokines, resulting in the necrosis of a large number of cells, and a severe fatal pneumonia is caused in the lungs.

The Anisomelic acid or Ovatodiolide in the present invention is prepared by extracting the whole plant, above-ground branches and leaves, or leaves of Anisomeles indica O. Kuntze with a solvent, and then separating and purifying by a column, wherein the solvent includes but not limits to water, methanol, ethanol, acetone, ethers, ethyl acetate, esters or hexane, and the column includes but not limits to alumina, silicon oxide, and silica gel columns. In addition, the Anisomelic acid or Ovatodiolide can also be prepared by chemical synthesis method.

The term “safe and effective amount” in the present invention refers to a safe and effective amount of Anisomelic acid or Ovatodiolide, or an amount of Anisomelic acid or Ovatodiolide with inhibitory or therapeutic effects and combinations of its pharmaceutically acceptable salts or carriers. The changes in safe and effective amount may vary depending on the route of administration, excipient usage, and co-usage with other active agents.

In the present invention, wherein the pharmaceutically acceptable salt can be formed into an appropriate pharmaceutical form with at least one solid, liquid or semi-liquid excipient or auxiliary agent, and wherein the appropriate pharmaceutical form includes but not limits to tablets, capsules, emulsions, aqueous suspensions, dispersions and solutions, etc.

The carrier used in the pharmaceutical composition of the present invention must be “acceptable”, which is compatible with the active ingredients of the formulation (and preferably have the ability to stabilize the active ingredients) and not harmful to the patient, for example, co-solvent cyclodextrins, which form a specific, more soluble complexes with the active compounds of one or more extract. Other examples of carriers used as pharmacological adjuvants for the delivery of active ingredients include colloidal silicon dioxide, magnesium stearate, cellulose and sodium lauryl sulfate, etc.

In the present invention, wherein the pharmaceutical composition is administered orally, parenterally, via inhalation spray or using an implanted reservoir.

The pharmaceutical composition of the present invention can also be prepared into an inhalation component according to well-known techniques in this technical field. For example, a salt solution that can be prepared by using benzyl alcohol or other suitable preservatives, adsorption promoters that enhance bioavailability, fluorocarbons, or other solubilizing or dispersing agents well known in the art.

In the present invention, the carriers generally used for tablets include lactose and corn starch, and lubricating agents, such as magnesium stearate, are also generally added to the tablets; the diluents used in capsule form generally include lactose and dried corn starch; when the oral administration is an aqueous suspension or emulsion, the active ingredient can be suspended or dissolved in the oily phase combined with the emulsifying or suspending agent; if necessary, specific sweetening, flavoring and coloring agents can also be added.

In one embodiment, the pharmaceutical compositions of the present invention may also be prepared to a sterile injectable preparation (e.g., aqueous or oily suspension). For example, using suitable dispersing or humidifying (e.g., Tween 80) agents and suspension by using well-known techniques in the art, the sterile injection preparation can also be that adding the sterile injection solution or suspension into a non-toxic parenteral diluent or solvent, such as into 1,3-Butanediol. The available vehicles and solvents include mannitol, water, Ringer's solution and isotonic sodium chloride solution. In another embodiment, the sterile injection preparation can also be that adding the sterile injection solution or suspension into non-toxic parenteral diluents or solvents, such as into 1,3-Butanediol. The available vehicles and solvents include mannitol, water, Ringer's solution and isotonic sodium chloride solution.

The sterile and fixed oils are often used as solvents or suspending vehicles (such as synthetic mono- or di-glycerides), and fatty acids, such as oleic acid and its glyceride derivatives can also be used in the preparation of injections, which are natural pharmaceutically acceptable oils, such as olive oil, castor oil, especially in their polyoxyethylated variations, these oil solutions or suspensions may also include diluents with long-chain alcohols or dispersant, or carboxymethyl cellulose or the similar dispersant.

Anisomelic acid or its oxidized derivative, Ovatodiolide, of the present invention competitively inhibits the infection of novel coronavirus (binding with human cell NRP1 receptor), inhibits virus invasion of cells (inhibits TMPRSS2), inhibits virus integration into cells (inhibits Cathepsin B/L) and inhibiting viral replication (inhibits Mpro) (FIG. 3); it illustrates that Anisomelic acid or Ovatodiolide is a potential small molecule drug that can inhibit the invasion of novel coronavirus infection and its replication in host cells; the model may provide new prevention and treatment strategies for COVID-19.

### BRIEF DESCRIPTION OF THE DRAWINGS

FIG. 1 illustrates the chemical structural formula of Anisomelic acid (AA), Formula I.

FIG. 2 illustrates the chemical structural formula of Ovatodiolide, Formula II.

FIG. 3 illustrates a summary schematic of the mechanism of the diterpenoid natural compounds of Anisomeles indica O. Kuntze, Anisomelic acid or Ovatodiolide, in inhibiting the infection and replication of novel coronavirus. Abbreviation: ACE2: Angiotensin-converting enzyme-2; NRP1: Neuropilin-1; TMPRSS2: Transmembrane serine protease; Cathepsin: Cathepsin; Main protease: SARS-CoV-2 virus Main protease; ppla/pplab: Polyprotein A/Polyprotein B; Nsps: Non-structural proteins; Nucleocapsid: Nucleocapsid protein.

FIGS. 4A-4B illustrate the molecular docking simulation study and biochemical analysis of inhibitory effect of Anisomelic acid (AA) in the present invention for competitively inhibiting the binding of the novel coronavirus spike protein S1 (SARS-CoV-2 spike S1) to the human Neuropilin-1 (NRP1) receptor. FIG. 4A. the docking structure of Anisomelic acid (AA) with the human NRP1, wherein the carboxyl group of AA forms hydrogen bonds with the side chain hydroxyl groups of Ser346, Thr349 and Tyr353 of NRP1. The hydrophobic alicyclic and the side chain bind to the hydrophobic groove of NRP1 formed by hydrophobic amino acids Tyr297, Trp301, Thr316, Lys351 and Tyr353. FIG. 4B. The Anisomelic acid binds to the human Neuropilin-1 (NRP1) receptor and competitively inhibits the novel coronavirus spike protein S1 (SARS-CoV-2 spike S1), resulting in a reduction in the probability of the binding of novel coronavirus spike protein S1 to the NRP1 receptor, that is, the infectious activity of the novel coronavirus is inhibited.

FIGS. 5A-5B illustrates the molecular docking simulation study and biochemical analysis of inhibitory effect of Anisomelic acid (AA) in the present invention binding to host cathepsin (Cathepsin B and Cathepsin L). FIG. 5A. the docking structure of Anisomelic acid (AA) with Cathepsin B and Cathepsin L, wherein AA forms covalent bonds with the catalytic cysteine C29 of Cathepsin B and the catalytic cysteine C25 of Cathepsin L using an exocyclic olefin, respectively. FIG. 5B. the inhibitory effect of Anisomelic acid on the activity of host cathepsin (Cathepsin B and Cathepsin L).

FIGS. 6A-6B illustrates the molecular docking simulation study and biochemical analysis of inhibitory effect of Anisomelic acid (AA) in the present invention binding to the host transmembrane serine protease S2 (TMPRSS2). FIG. 6A. the docking structure of Anisomelic acid with TMPRSS2, wherein the hydrophobic alicyclic and the side chain of AA bind to the hydrophobic amino acids Val280 and Leu302 of TMPRSS2 to form a hydrophobic groove, and a stable compound is formed through electrostatic interactions of the carboxyl group of Anisomelic acid and Lys342. FIG. 6B. the inhibitory effect of Anisomelic acid on the activity of the host TMPRSS2.

FIGS. 7A-7B illustrates the molecular docking simulation study and biochemical analysis of inhibitory effect of Anisomelic acid (AA) with the main protease (Mpro) of the SARS-CoV-2 virus. FIG. 7A. The docking structure of AA with the main protease (Mpro) of the SARS-CoV-2 virus, wherein the lactone carbonyl group of AA forms hydrogen bonds with the main chain N—H of Gly143, Ser144, and Cys145 of Mpro, the exocyclic double bond forms a covalent bond with the catalytic amino acid Cys145, the hydrophobic alicyclic and the side chain bind to the hydrophobic amino acids Thr25, Leu27, Cys44 and Met49 of Mpro to form a hydrophobic groove through the hydrogen bonding between carboxyl group and Gln189. FIG. 7B. the inhibitory effect of Anisomelic acid on the activity of the main protease (Mpro) of the novel coronavirus.

FIG. 8 illustrates a simulation example of the inhibitory effect of Anisomelic acid (AA) in the present invention on a series of novel coronavirus variants. The binding site of SARS-CoV-2 spike S1 and the human NRP1 receptor, which is competitively inhibited by Anisomelic acid, belongs to a highly conserved site due to the involvement of furin cleavage site. According to the statistics of the Global Initiative on Sharing All Influenza Data (GISIAD), the conservation of this target is 100%.

FIGS. 9A-9C illustrate the results of animal experiments for comparing the inhibitory effects of SARS-CoV-2 virus infection of the Anisomelic acid and its oxidized derivative, Ovatodiolide, in the present invention and the antiviral drug Remdesivir in virus-susceptible K18-hACE2 transgenic mice. FIG. 9A. the experimental execution, schedule, drug administration methods, dosage, and other planning for the animal experiments. FIG. 9B. the daily weight changes of the transgenic mice after virus challenge and drugs administration. FIG. 9C. the changes in the viral titers of the coronavirus infection in lungs of the transgenic mice.

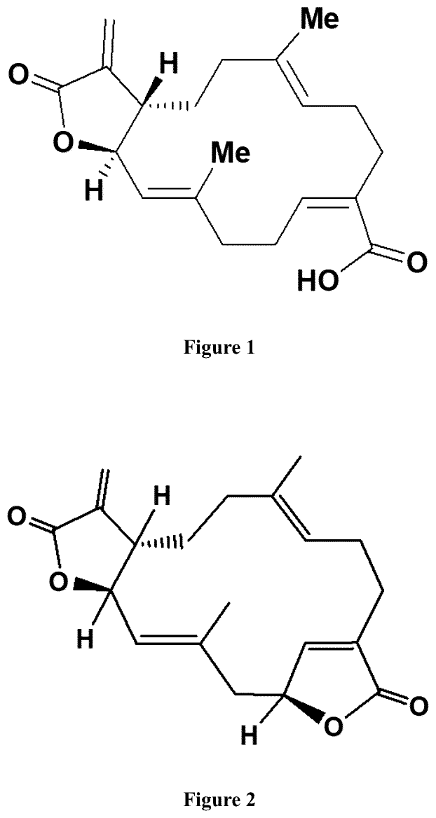

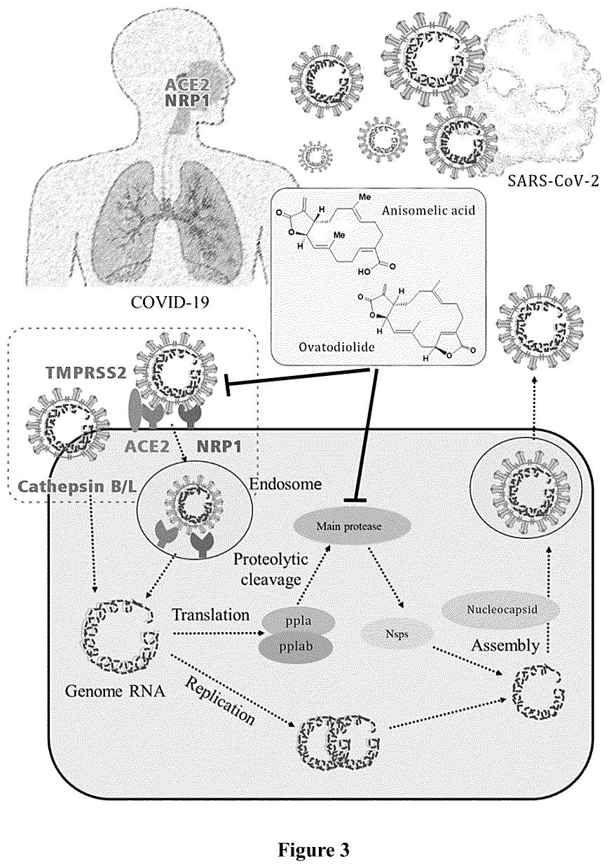

### EMBODIMENTS

In order to better understand the technical content of the present invention to the potential technical implementers, the following specific embodiments are hereby cited. The following embodiments are only used to explain the present invention, but the claimed scope of the present invention is not limited to the following embodiments.

In order to make the above purpose, features, and advantages of the present invention more apparent and comprehensible, the following embodiments are presented in a systematic and focused manner.

In the section regarding the mechanism of action of compound molecules in the embodiments, Anisomelic acid was specifically chosen as the test sample for comprehensive elucidation. Additionally, the used test sample in the present invention on the inhibitory activity of infection of host cells by a series of the novel coronavirus variants and the comparative animal experiments on the inhibitory effects of SARS-CoV-2 virus infection in virus-susceptible K18-hACE2 transgenic mice included Anisomelic acid and its oxidized derivative Ovatodiolide as well as the positive control antiviral drug, Remdesivir.

### Example 1: The Molecular Docking Simulation Study and Biochemical Analysis of the Inhibitory Effects of Anisomelic Acid (AA) on the Competitive Binding of the SARS-CoV-2 Spike S1 Protein to the Neuropilin-1 (NRP1) Receptor of Human Cells

The Neuropilin-1 (NRP1) receptor on the human cells surface, which is an important target in antiviral drug research, mediates the viral infection process by binding to the activated spike protein (S protein) of the SARS-CoV-2 virus. The embodiment is based on molecular docking to evaluate whether Anisomelic acid binds to and inhibits the Neuropilin-1 (NRP1) and it mediates SARS-CoV-2 infection process, and thereby to elucidate the antiviral mechanism of Anisomelic acid.

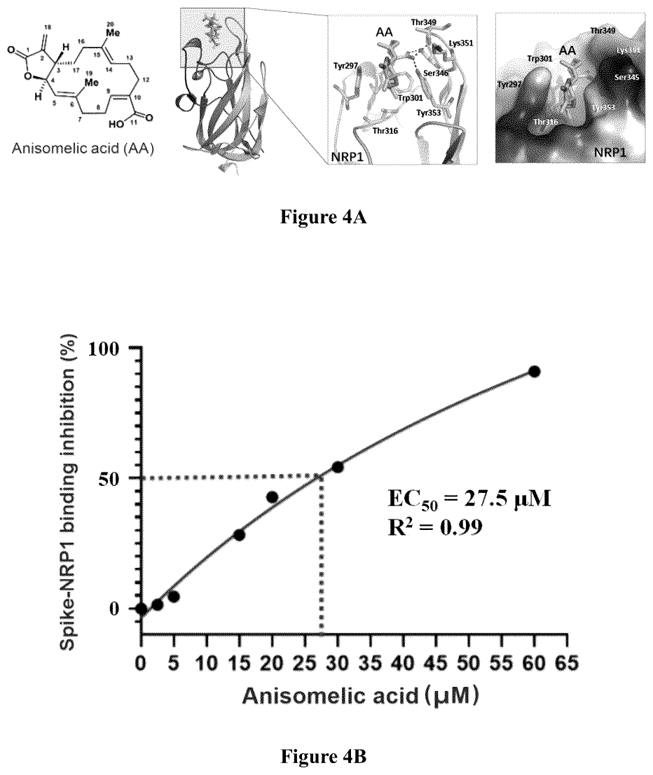

The specific embodiment was as follows: the crystal structure of Neuropilin-1 (NRP1) (PDB code: 2ORZ), a human cell surface receptor was used as molecular docking receptor, hydrogen atoms was added to the structure of NRP1 by MOE (Molecular Operating Environment) software and energy optimization was performed. The structure of the ligand Anisomelic acid (AA) was also constructed by MOE software, and the energy optimization was performed by using the standard MMFF94 molecular force field and the energy gradient of 0.0001 kcal/mol as the convergence criteria. The molecular docking was carried out based on the molecular docking module of MOE, the energy optimization and docking mode analyzation of the energy-optimal docking structure were further conducted. The results of the molecular docking showed that Anisomelic acid (AA) could bind to the ligand binding pocket of NRP1. As show in FIG. 4A, the carboxyl group of AA formed hydrogen bonds with the side chain hydroxyl groups of Ser346, Thr349 and Tyr353 of NRP1, and the hydrophobic alicyclic and the side chains bound to the hydrophobic groove formed by the hydrophobic amino acids Tyr297, Trp301, Thr316, Lys351 and Tyr353 of NRP1. Based on the docking results, it was predicted that Anisomelic acid (AA) could block the binding of NRP1 to the viral spike protein by binding to the ligand binding pocket of NRP1, thereby inhibiting the infection of SARS-CoV-2.

In the biochemical experiments, the competitive inhibition of Anisomelic acid on binding of the novel coronavirus spike glycoprotein S1 to the human Neuropilin-1 (NRP1) receptor was confirmed by enzyme-linked immunosorbent assay (ELISA): for the indirect ELISA, 100 μg receptor protein was coated on a Nunc® MaxiSorp™ 96-well plates by using a concentration of 100 mM carbonate buffer, and protein blocking was performed by using gelatin buffer. According to the manufacturer's instructions, the monoclonal antibody of SARS-CoV-2 Spike S1 (Catalog No: E-AB-V1005) was diluted at 1:200 dilution and tested the linked HRP or AlexaFluor488 as a biomarker. For the competitive ELISA, a concentration of 5 g/mL human Neuropilin-1 (Elabscience) or human ACE2 (Elabscience) recombinant proteins were coated on Nunc® MaxiSorp™ 96-well plates, incubated overnight at 4° C., and protein blocking was carried out using gelatin buffer at 37° C. In the control group, different concentrations (0, 2.5, 5, 10, 20, 30 μM) of Anisomelic acid and fixed concentration of 20 μg of recombinant novel coronavirus S1 protein were added to test the effect of Anisomelic acid blocking the binding of S1 to the receptor, and then were incubated for 2 hours at 37° C. After thorough washing, the monoclonal antibody of SARS-CoV-2 Spike S1 in diluted 1:1000 was added, followed by incubation for 1 hour at 37° C. After the addition of TMB substrate (Invitrogen) and stop solution (KPL SeraCare), colorimetric reactions were quantified. The absorbance of the samples was measured at 450 nm, and the background values were measured at 570 nm. As shown in FIG. 4B, the result indicated that the EC₅₀ of Anisomelic acid for inhibiting the binding of the novel coronavirus to the NRP1 receptor was approximately 27.5 μM.

### Example 2: Molecular Docking Simulation and Biochemical Analysis of the Inhibitory Effect on the Binding of Anisomelic Acid to the Host Endosomal Cysteine Proteolytic Enzymes Cathepsin B and Cathepsin L

Cathepsin B is a cysteine protease that primarily plays a role as a protease in lysosomes of normal cells and plays a role in protein degradation. Aberrant expression of high level Cathepsin has been observed in various human cancers and experimental models, such as transgenic models of murine pancreatic cancer and breast cancer, and it has been confirmed that this protease plays an important role in initiation, growth, proliferation, angiogenesis, and invasion of tumor cells. Cathepsin B is mainly involved in the degradation of lysosomal proteins. In addition to its effect in the protein cycle, Cathepsin B is involved in the infection cycles of several viruses including Ebola virus, Nipah virus, Moloney murine leukemia virus, and feline coronavirus. Cathepsin B has the ability to catalyze and activate the viral membrane glycoproteins, which results in the release of viruses from the endosomal to the cytoplasm through the fusion of the virus envelope with the endosomal membrane.

Cathepsin L is a cysteine protease located within the cell lysosomes and involves in many fundamental physiological processes including the degradation and renewal of intracellular proteins, antigen presentation, and organ development. Cathepsin L has been known to play an important role in tumor metastasis and chemotherapy resistance, and abnormal expressions of this enzyme have been found in a variety of cancers. In the infection mechanism of respiratory viruses (such as influenza virus), cathepsin L is an important key in the process of virus invading host lysosomes, triggering subsequent viral infection by cleaving viral antigens. In the traditional mechanism of SARS-CoV-2 virus invasion of the host, Cathepsin L also plays a vital role in activating the viral spike protein antigen in lysosomes. It also plays an important role in the activation of viral spike antigens within lysosomes in the traditional mechanism of host invasion of SARS-CoV-2 virus. The further infection of novel coronavirus can be prevented by inhibiting the activity of Cathepsin L. In summary, the host endosomal cysteine proteolytic enzymes Cathepsin B and Cathepsin L play a crucial role in the fusion process of coronaviruses.

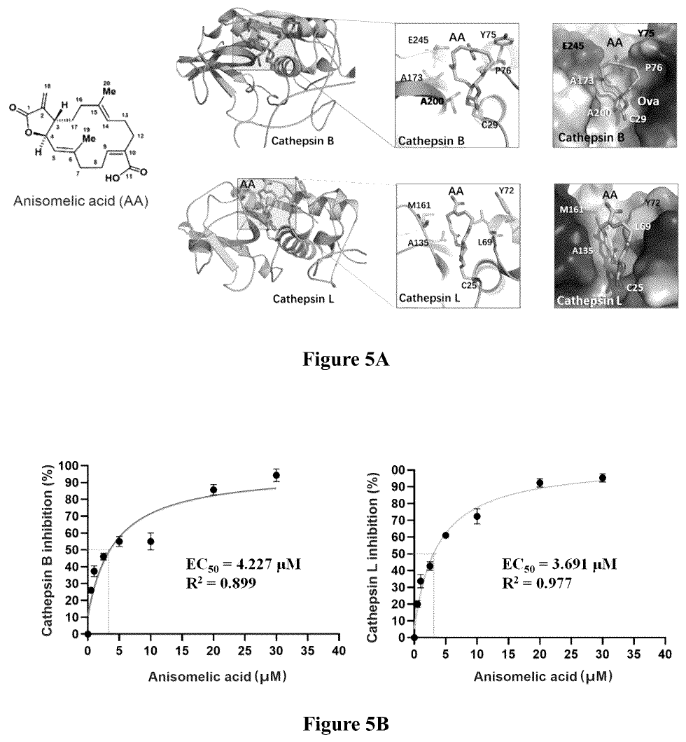

The embodiment was based on molecular docking to evaluate whether Anisomelic acid bound and inhibits Cathepsin B and Cathepsin L to elucidating the anti-novel coronavirus mechanism of Anisomelic acid. The embodiment was as follows: the crystal structures of human cysteine proteolytic enzymes Cathepsin B and Cathepsin L (PDB codes: 3A18 & 2XU1), were used as molecular docking receptors, respectively, and the hydrogen atoms were added to the structures of Cathepsin B and Cathepsin L by MOE software and then energy optimization were carried out. The structure of ligand Anisomelic acid (AA) was constructed by MOE software, and the energy optimization of convergence criteria was performed by using the standard MMFF94 molecular force field and the energy gradient of 0.0001 kcal/mol. The molecular docking was carried out based on the molecular docking module of MOE, the energy optimization and docking mode analyzation of the energy-optimal docking structure were further conducted. The results of the molecular docking indicate that Anisomelic acid (AA) was able to bind to the catalytic pocket of endosomal cysteine proteolytic enzymes Cathepsin B and Cathepsin L as well. AA bound to the hydrophobic S2 site composed by Y75, P76, A173, A200 and E245 in Cathepsin B by a hydrophobic alicyclic and inhibited the activity of Cathepsin B through a covalent complex formed by the exocyclic olefin and the catalytic cysteine C29 (see FIG. 5A). On the other hand, AA bound to the hydrophobic S2 site of Cathepsin L consisting of L69, M70, Y72, A135 and M161 with a hydrophobic alicyclic and inhibited the activity of Cathepsin L through a covalent complex formed by the exocyclic olefin and catalytic cysteine C25. Since the endosomal cysteine proteolytic enzymes Cathepsin B and Cathepsin L played a crucial role in the fusion process of coronaviruses, AA had the potential to block the invasion and fusion process of the novel coronavirus.

In the biochemical test, the Cathepsin B Fluorescence Assay Kit (Cathepsin B Inhibitor Screening Assay Kit, BPS BIOSCIENCE, 79590) was used in the test method. According to the manufacturer's manual, the inhibition activity was measured through continuous kinetic analysis, the samples containing Cathepsin B were reacted with a fluorescent substrate in the mixing, and the measured fluorescence (λex=360 nm, λem=460 nm) readings were used. The E64 protease inhibitor was used as a positive control. The experiment was conducted in a 50 μL reaction system, with a single reaction dose of Cathepsin B at 0.4 ng/group, the E64 protease inhibitor were used as a positive control. The inhibitory impact of various concentrations of Anisomelic acid (0.5, 1, 2.5, 5, 10, 20, and 30 μM) against Cathepsin B protease activity was examined. The significant inhibition of Anisomelic acid against Cathepsin B protease activity was found in a dose-dependent manner. The half maximal effective concentration (EC₅₀) of Anisomelic acid was calculated to be 4.227 μM. These results indicated that Anisomelic acid is a potential inhibitor of Cathepsin B protease as shown in the left graph of FIG. 5B.

The Cathepsin L Fluorescence Assay Kit (Cathepsin L Inhibitor Screening Assay Kit, BPS BIOSCIENCE, 79591) was used in the biochemical test method. According to the manufacturer's manual, the inhibition activity was measured through continuous kinetic analysis, the samples containing Cathepsin L were reacted with a fluorescent substrate in mixing, and the measured fluorescence (λex=360 nm, λem=460 nm) readings were used. The E64 protease inhibitor was used as a positive control. The experiment was conducted in a 50 μL reaction system, with a single reaction dose of Cathepsin L at 0.4 ng/group, the E64 was used as the positive control of inhibition. The inhibitory impact of various concentrations of Anisomelic acid (0.5, 1, 2.5, 5, 10, 20, and 30 μM) against Cathepsin L protease activity was examined. As shown in the right graph of FIG. 5B, the significant inhibition of Anisomelic acid against Cathepsin L protease activity was found in a dose-dependent manner. The half maximal effective concentration (EC₅₀) of Anisomelic acid was calculated to be 3.691 μM. These results indicated that Anisomelic acid is a potential inhibitor of Cathepsin L protease.

### Example 3: Molecular Docking Simulation Study and Biochemical Analysis of Inhibitory Effects on the Binding of Anisomelic Acid to the Host Transmembrane Serine Protease (TMPRSS2)

The transmembrane protease TMPRSS2 is an enzyme belonging to the serine protease family and is also an important target of prostatic cancer. In the infection mechanism of the mutated strains of SARS-CoV-2 (such as B.1.617.2, Delta), viral spike protein S1/S2 can be greatly cleaved through TMPRSS2, then subsequent rapid invasion and infection were triggered, and the invasion of host cells by SARS-CoV-2 viral particles was promoted. This new mechanism greatly improves the infection efficiency of the novel coronavirus mutant strains. The viral spike protein which combined with the human cell surface receptor angiotensin-converting enzyme 2 (ACE2) could be prevented from continuing to invade cells rapidly by inhibiting TMPRSS2, thereby the integration of the SARS-CoV-2 virus into host cells was limited. This mechanism makes TMPRSS2 become an important novel therapeutic target. SARS-CoV-2 differs from SARS-COV in that it efficiently uses TMPRSS2, an enzyme found in abundance outside respiratory cells. First, TMPRSS2 cut at the S2 subunit of the spike. This cut exposed a series of hydrophobic amino acids. Then, the elongated spike protein folded back on itself like a zipper, and forced the fusion of virus and cell membranes. In view of the speed of virus mutation, competitive inhibition of the host targets may be the goal of the development of novel small molecule drugs in the future. In summary, transmembrane serine proteases (TMPRSS2) in human cells plays a key role in the invasion process of SARS-CoV-2 virus by cleaving and activating the spike protein.

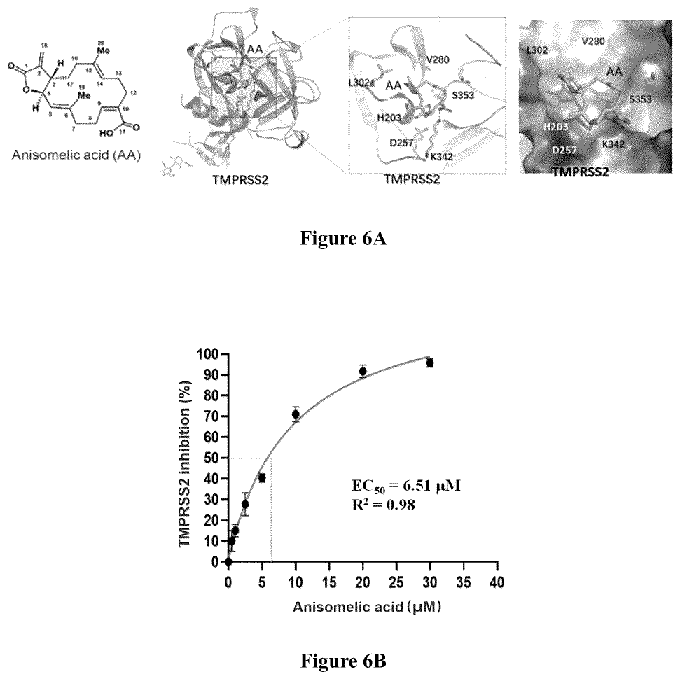

The example was to evaluate whether Anisomelic acid bound and inhibited TMPRSS2 based on molecular docking to elucidate the antiviral mechanism of Anisomelic acid. The specific embodiment was as follows: the crystal structure of TMPRSS2 (PDB code: 7MEQ) in human cells was used as the molecular docking receptor, and the hydrogen atoms were added to the structures of TMPRSS2 by MOE software and then energy optimization were carried out. The structure of ligand Anisomelic acid (AA) was constructed by MOE software, and the energy optimization of convergence criteria was performed by using the standard MMFF94 molecular force field and the energy gradient of 0.0001 kcal/mol. The molecular docking was carried out based on the molecular docking module of MOE, the energy optimization and docking mode analyzation of the energy-optimal docking structure were further conducted. The results of the molecular docking indicated that Anisomelic acid (AA) was able to bind to the active pocket of TMPRSS2. The hydrophobic alicyclic and side chain of AA bound to the hydrophobic groove formed by the hydrophobic amino acids Val280 and Leu302 of TMPRSS2, a stable compound was formed through the electrostatic interaction between the carboxyl group and Lys342 (FIG. 6A). Based on the docking results, it was predicted that Anisomelic acid (AA) could block the binding of TMPRSS2 to the substrate SARS-CoV-2 viral spike protein by binding to the active pocket of TMPRSS2, thereby inhibiting the invasion process of SARS-CoV-2.

In the biochemical experiments, TMPRSS2 Fluorescence Assay Kit (TMPRSS2 Fluorogenic Assay Kit, BPS BIOSCIENCE, 78083) was used in the test method. Inhibitory activity was measured by sequential kinetic analysis according to the manufacturer's manual, the inhibition activity was measured by using continuous kinetic analysis, the samples containing TMPRSS2 were reacted with a fluorescent substrate in a mixing, and a fluorescence (λex=383 nm, λem=455 nm) readings were used. Camostat protease inhibitor was used as a positive control. The experiment was performed in a 50 μL reaction system, the single reaction dose of TMPRSS2 was 150 ng/group, and 10 μM Camostat protease inhibitor was used as a positive control. The effect of Anisomelic acid on the inhibition of TMPRSS2 protease activity was examined by tested various concentrations of Anisomelic acid (0.5, 1, 2.5, 5, 10, 20 and 30 μM). A significant inhibition of Anisomelic acid against TMPRSS2 protease activity was found in a dose-dependent manner. The half maximal effective concentration (EC₅₀) of Anisomelic acid was calculated to be 6.51 μM. These results indicate that Anisomelic acid is a potential novel inhibitor of TMPRSS2 protease (FIG. 6B).

### Example 4: Molecular Docking Simulation Study and Biochemical Analysis of Inhibitory Effect on the Binding of Anisomelic Acid to SARS-CoV-2 Viral Main Protease (Mpro)

Novel coronaviral main protease (Mpro), which is also known as 3CLpro, can regulate the program of the coronavirus replication complex. It is a cysteine protease that participates in cleavage step of the viral polyproteolytic and ultimately forms a series of functional proteins required for coronavirus replication, which is currently an effective target for designing anti-SARS drugs. In conclusion, the main protease of SARS-CoV-2 (Mpro) is a key SARS-CoV-2 enzyme that plays a key role in mediating viral replication and transcription and is an attractive drug target for the virus.

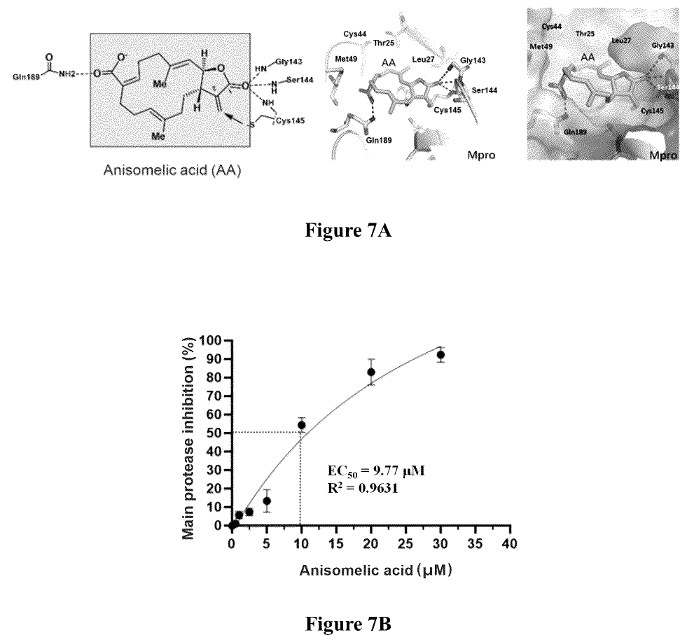

This example was to evaluate whether Anisomelic acid bound and inhibited the SARS-CoV-2 viral main protease (Mpro) based on molecular docking to elucidate the antiviral mechanism of Anisomelic acid. The specific embodiment was as follows: the crystal structure of SARS-CoV-2 main protease (Mpro) (PDB code: 6Y2G) was used as the molecular docking receptor, hydrogen atoms were added to the Mpro structure by using MOE software and then energy optimization were carried out. The structure of ligand Anisomelic acid (AA) was constructed by MOE software, and the energy optimization of convergence criteria was performed by using the standard MMFF94 molecular force field and the energy gradient of 0.0001 kcal/mol. The molecular docking was carried out based on the molecular docking module of MOE. the energy optimization and docking mode analyzation of the energy-optimal docking structure were further conducted. The results of the molecular docking indicated that Anisomelic acid (AA) was able to bind to the active pocket of Mpro. The lactone carbonyl group at position 1 of AA formed a hydrogen bond with the main chain N—H of Gly143, Ser144 and Cys145 of Mpro, the exocyclic double bond formed a covalent bond with the catalytic amino acid Cys145, the hydrophobic alicyclic and side chain bound to the hydrophobic amino acids Thr25, Leu27, Cys44 and Met49 of Mpro to form a hydrophobic groove through the hydrogen bonding between the lactone carbonyl or carboxyl group and Gln189 (FIG. 7A). According to the docking results, it is predicted that Anisomelic acid (AA) was predicted to be able to inhibit the replication of SARS-CoV-2 by binding to the active pocket of Mpro and blocking the binding of Mpro to the matrix.

In the biochemical experiments, a recombinant 2019-nCoV 3CL protease protein (Elabscience bio Inc) was used. Catalytic activity was measured by continuous kinetic analysis, the same fluorescent substrate Dabcyl-KTSAVLQSGFRKME-Edans (from synthetic) was used as substrate and the fluorescent signal generated due to the protease-catalyzed substrate lysis was tested, the signal was read at 538 nm, the excitation wavelength was 355 nm. The experiment was performed in a 100 μL reaction system, and the buffer was consisted of 50 mM Tris. HCl (pH 7.3) and 1 mM ethylenedinitrilotetraacetic acid. To measure the EC₅₀ of the compound, 500 nM enzyme, 20 μM substrate, and seven different concentrations of Anisomelic acid were added into different wells. Compounds were dissolved and diluted in dimethyl sulfoxide to the desired concentration. 1 μL diluted compound was added into 50 μL solution containing 1 μM Mpro, and then the solution was left at room temperature for 10 min. 50 μL matrix was added to start the reaction, And Fluorescent intensity was monitored every 45 seconds. The initial reaction rate was calculated by fitting the linear part of the curve (within the first 5 min of the progression curve) into a straight line by using the SoftMax Pro program and converted to enzyme activity (substrate cleavage)/second. The effect of Anisomelic acid on the inhibition of Mpro activity was examined by testing various concentrations of Anisomelic acid (0.5, 1, 2.5, 5, 10, 20 and 30 μM). See FIG. 7B, the significant inhibition of Anisomelic acid against Mpro activity was found in a dose-dependent manner. The half maximal effective concentration (EC₅₀) of Anisomelic acid was calculated to be 9.77 μM, And the results indicated that Anisomelic acid was an inhibitor of the SARS-CoV-2 main protease (Mpro).

### Example 5: Simulation Example of the Inhibition of Anisomelic Acid Against a Series of Novel Coronavirus Mutant Strains

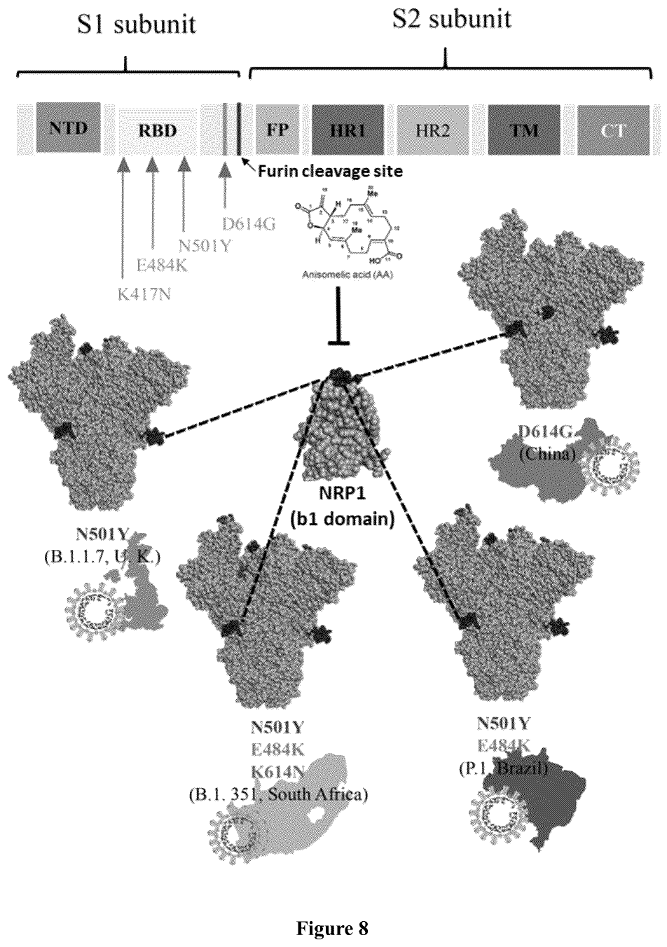

The purpose of the example was to observe and mark the mutation sites on the S1 structure of the current major novel coronavirus mutant strains in the world, and thereby to confirm whether Anisomelic acid has broad significance against viral invasion of hosts for the competitive inhibition of the binding site of SARS-CoV-2 spike S1 and the human NRP1 receptor. The point mutation positions of SARS-CoV-2 S protein were marked by using Discovery Studio software (Dassault Systemes BIOVIA, U.S.). The molecular structures were visualized and analyzed by using UCSF (University of California, San Francisco) Chimeram software. Relevant protein structure information was obtained from the Protein Database (https://www.rcsb.org/). The reference virus strains included dominant strains currently circulating internationally, including: D614G mutant strain (China), B.1.1.7 (UK), B.1.351 (South Africa), and P.1. (Brazil). The positions of point mutation of the above virus strains were marked on the protein structure, and the binding sites of the SARS-CoV-2 S protein and the human NRP1 receptor were compared. As shown in FIG. 8, the results of protein structure calibration showed that most of the mutation positions of the major virus strains currently circulating in the world were located at the receptor binding site RBD position of the spike protein, which adapted to the ability to bind to the host ACE2 receptor. And, the binding site of SARS-CoV-2 spike S1 to the human NRP1 receptor, which was competitively inhibited by Anisomelic acid, was a highly conserved site since it involved furin cleavage site. According to the statistics of Global Initiative on Sharing All Influenza Data (GISIAD), the conservation of this target is 100%. According to the above results and observations, Anisomelic acid competitively inhibited the binding site of SARS-CoV-2 spike S1 to human NRP1 receptor and had cross-virus strain and broad application value in inhibiting infection of viruses and invasion of host.

### Example 6: Studies on the Activity of Anisomelic Acid and its Oxidized Derivative Ovatodiolide in Inhibiting the Infection of Host Cells by the Novel Coronavirus and a Series of Virus Mutant Strains

According to the results of the molecular docking simulation studies and biochemical analyses between Anisomelic acid and the host cell endosomal cysteine proteolytic enzymes Cathepsin B and Cathepsin L described in example 2, as well as the results of the molecular docking simulation studies and biochemical analyses between Anisomelic acid and transmembrane protease serine 2 (TMPRSS2) on the surface of host cells, both predicted that Anisomelic acid had the ability to inhibit the host cell infection process of the novel coronavirus.

The example was based on the novel coronavirus pseudovirus activity inhibition detecting system developed by the laboratory of Professor Zhang Linqi, director of the AIDS Comprehensive Research Center of Tsinghua University, Beijing, to evaluate whether Anisomelic acid blocked the host cell infection process of novel coronavirus.

The specific embodiment was carried out in two steps based on the construction of novel coronavirus pseudovirus and the detection of inhibition of novel coronavirus infection: Step 1. 293T cells were co-transfected with membrane glycoprotein deleted (Env-defective) and luciferin expressed HIV-1 viral genome plasmid pNL4-3R-E-luciferase and the plasmid pcDNA3.1/SARS-CoV-2 with the expression of the full-length surface spike glycoprotein of the novel coronavirus, and then the 293T cells were cultured in DMEM medium containing 10% fetal bovine serum for 60 hours. The culturing supernatant was used to obtain the virus solution of the novel coronavirus pseudovirus (referred to SARS-CoV-2 virus solution). Step 2. 96-wells cell culture plate was used, 100 μL Anisomelic acid diluted solution and 50 μL SARS-CoV-2 virus solution were added into each well (the viral concentration in 50 μL SARS-CoV-2 virus solution was 1×10⁴ TCID50/mL) to make the concentration of Anisomelic acid diluted solution in the mixed system was the corresponding diluted concentration, and incubated at 37° C. for 1 hour. An equal volume of DMEM culture medium containing 10% fetal bovine serum was used to replace the Anisomelic acid diluted solution as a virus control. An equal volume of DMEM medium containing 10% fetal bovine serum was used to replace the SARS-CoV-2 virus solution as a cell control. The cell culture plate was used, 100 μL of Huh7 cell suspension (the solvent used to prepare the cell suspension was DMEM medium containing 10% fetal bovine serum, and the Huh7 cell concentration in the cell suspension was 2×10⁵ cells/mL) was inoculated into each well and was incubated at 37° C. for 64 hours. The supernatant was aspirated and discarded, 150 μL of lysis buffer (Vigorous Biotechnology, Cat. No. T003, according to the instructions) was added into each well, and incubated at 37° C. for 5 minutes. The cell culture plate was taken for the detection of luciferase activity. Multiple wells were set up for each treatment. Inhibitory activity (%)=[1−(fluorescence intensity of test group−fluorescence intensity of cell control)/(fluorescence intensity of virus control−fluorescence intensity of cell control)]×100%. The concentration of Anisomelic acid at 50% inhibitory activity, namely the IC₅₀ value of Anisomelic acid, was calculated by using Prism 5 software.

In the above experiments, pseudoviruses were constructed for the Wuhan virus strain and seven other virus variants, and a series of pseudovirus infection inhibition tests were conducted respectively. The names of the novel coronaviruses and variants used in this experiment were as follows: Wuhan D614, South Africa B.1.351, Brazil P.1, India B.1.617.1, Uganda A23.1, Nigeria B.1.525, California B.1.429, New York B.1.526 etc.

The series of IC₅₀ values of Anisomelic acid and Ovatodiolide agains eight novel coronavirus strains mentioned above, compared with the series of IC₅₀ values of antiviral drug Remdesivir, as shown in Table 1. The research results showed that both Anisomelic acid and Ovatodiolide had similar inhibitory effects on novel coronavirus infection at the micromolar level to Remdesivir.

TABLE 1
Individual half-inhibitory concentrations of compounds such as
Anisomelic acid, Remdesivir, and Ovatodiolide in inhibiting
infection of eight novel coronavirus mutant strains.

| SARS-COV-2 | Anisomelic acid | Remdesivir | Ovatodiolide |
|---|---|---|---|
| Variants | IC₅₀ (μg/mL) | IC₅₀ (μg/mL) | IC₅₀ (μg/mL) |
| Wuhan D614 | 3.50 | 2.96 | 4.08 |
| South Africa | 27.55 | 27.25 | 15.42 |
| B.1.351 | | | |
| Brazil P.1 | 0.12 | 13.75 | 7.00 |
| India B.1.617.1 | 10.09 | 11.91 | 15.27 |
| Uganda A23.1 | 15.53 | 7.63 | 6.72 |
| Nigeria B.1.525 | 14.36 | 10.31 | 8.24 |
| California B.1.429 | 8.47 | 15.91 | 12.09 |
| New York B.1.526 | 8.86 | 15.00 | 11.35 |

### Example 7: Comparative Animal Examination on the Inhibition of Anisomelic Acid and its Oxidized Derivative Ovatodiolide Against the Infection of SARS-CoV-2 Virus to K18-hACE2 Transgenic Mice

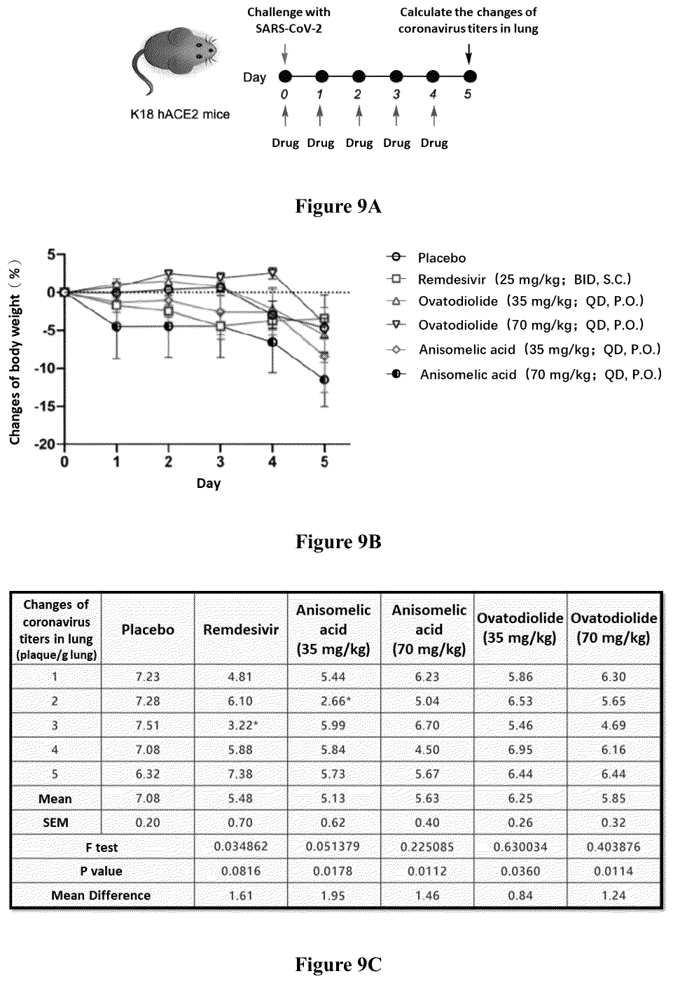

The present invention was specially commissioned by Nantong WuXi AppTec Co., Ltd., and was carried out professionally and concretely through the company's U.S. biological laboratory; K18-hACE2 transgenic coronavirus-susceptible mice were used to carry out the anti-SARS-CoV-2 animal experiments planned by the inventor's team. Six groups were devided in the experiment, and five mice were involved in each group, namely the blank group (1 group), the Remdesivir control group (1 group) and the natural products of Anisomeles indica O. Kuntze group (4 groups), with total of 30 experimental mice. Coronavirus (SARS-CoV-2) were were inoculated intranasally into mice at amounts of 1×10⁵ pfu. Mice were orally administered with Anisomelic acid, Ovatodiolide, or placebo one hour before challenge. The two experimental dosages of miceorally administered Anisomelic acid or Ovatodiolide every day were 35 mg/kg body weight and 70 mg/kg body weight respectively. The operation was continued for 4 days (the used dosages were converted into human applicable dosages with approximately 3 mg/kg-body weight and 6 mg/kg-body weight). The experiment was based on remdesivir, which was injected twice a day, 25 mg/kg each time. At the fifth day after the experiment, all experimental mice were sacrificed (FIG. 9A). The lung infection statuses of experimental mice were characterized by changes in coronavirus titers in the lungs and histopathological analyses.

The experimental results showed that: 1. In addition to the significant changes in body weight of the mice group administered placebo, the changes in body weight of the mice caused by administration of Anisomelic acid or Ovatodiolide and Remdesivir were basically similar (FIG. 9B), which could be described as the safety of the three compounds were similar; 2. Changes in the titers of coronavirus infection in the lungs of mice, showed that the changes in virus infected titers in mice of the two tested compounds, Anisomelic acid and Ovatodiolide, were similar to the virus infected titers in mice caused by Remdesivir (FIG. 9C). All of them showed prevention and control effects; wherein Anisomelic acid showed better prevention and control effects. The above results showed that Anisomelic acid and Ovatodiolide had the potential to be developed as oral anti-novel coronavirus drugs.

1. A method for inhibiting infection and replication of novel coronavirus in a subject suffering from invasion of novel coronavirus, comprising administering a composition comprises a safe and effective amount of Anisomelic acid, or a safe and effective amount of a structural isomer of Anisomelic acid, or a safe and effective amount of a derivative of Anisomelic acid to the subject, wherein the Anisomelic acid comprises a chemical structure of Formula I:

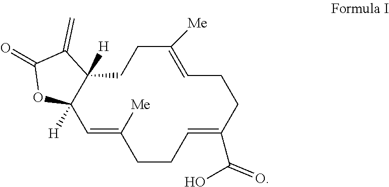 [SMILES: C=C1C(=O)O[C@@H]2/C=C(\C)CC/C=C(\C(=O)O)CC/C=C(\C)CC[C@@H]12]

2. The method of claim 1, wherein the composition further comprises a pharmaceutically acceptable salt or carrier of the Anisomelic acid, or the structural isomer of Anisomelic acid, or the derivative of Anisomelic acid.

3. The method of claim 1, wherein the composition is used for the prevention or treatment of diseases caused by the novel coronavirus.

4. The method of claim 1, wherein the composition is used for the prevention or treatment of diseases caused by novel coronavirus variants.

5. The method of claim 1, wherein the composition is used for the prevention or treatment of severe special infectious pneumonia.

6. The method of claim 1, wherein the Anisomelic acid is a natural compound prepared by extracting Anisomeles indica O. Kuntze with an organic solvent and then separating and purifying by a chromatography column, or an artificially synthesized compound with a structure of natural substrate of Anisomeles indica O. Kuntze prepared by a chemical synthesis method.

7. The method of claim 1, wherein the safe and effective amount is 180 mg to 360 mg orally and daily administered to a normal adult with a body weight of 60 kg and continuously administered for 3 to 5 days.

8. A method for inhibiting infection and replication of coronavirus in a subject suffering from invasion of coronavirus, comprising administering a composition comprises Anisomelic acid, or a structural isomer of Anisomelic acid, or a derivative of Anisomelic acid to the subject, wherein the Anisomelic acid comprises a chemical structure of Formula I:

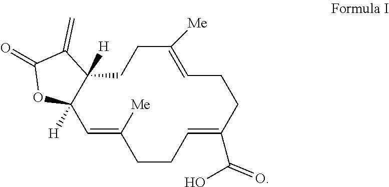 [SMILES: C=C1C(=O)O[C@@H]2/C=C(\C)CC/C=C(\C(=O)O)CC/C=C(\C)CC[C@@H]12]

9. A method for inhibiting infection and replication of novel coronavirus in a subject suffering from invasion of novel coronavirus, comprising administering a composition comprises a safe and effective amount of Anisomelic acid oxidized derivative Ovatodiolide, or a safe effective amount of structural isomer of the Ovatodiolide to the subject, wherein the Ovatodiolide comprises a chemical structure of Formula II:

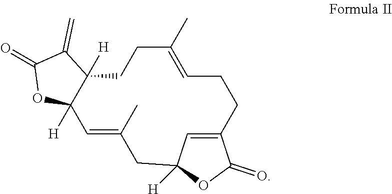 [SMILES: C=C1C(=O)O[C@@H]2/C=C(\C)C[C@H]3C=C(CC/C=C(\C)CC[C@@H]12)C(=O)O3]

10. The method of claim 9, wherein the composition further comprises the Ovatodiolide or the structural isomer of the Ovatodiolide and their pharmaceutically acceptable salts or carriers thereof.

11. The method of claim 9, wherein the oxidized derivative of Anisomelic acid, Ovatodiolide, is a natural compound prepared by extracting Anisomelic acid with an organic solvent and then separating and purifying by a chromatographic column, or an artificially synthesized compound with a structure of the natural substance of Ovatodiolide by a chemical synthesis method.

12. The method of claim 9, wherein the safe and effective amount is 180 mg to 360 mg orally and daily administered to a normal adult with a body weight of 60 kg and continuously administered for 3 to 5 days.
